(19) United States
(12) Patent Application Publication
(10) Pub. No.: US 2004/0267144 A1
(43) Pub. Date: Dec. 30, 2004

(54) Electrocardiogram signal converter and analog-to-digital conversion device thereof

(76) Inventors: Terry B. J. Kuo, Ji-An Township (TW); Cheryl C. H. Yang, Ji-An Township (TW)

Correspondence Address: John S. Egbert, Harrison & Egbert, 7th Floor, 412 Main Street, Houston, TX, 77002, US

(21) Appl. No.: 10/778,476

(22) Filed: Feb. 13, 2004

(30) Foreign Application Priority Data
Jun. 25, 2003 (TW) 092117250

(51) Int. Cl. A61B 5/0402

(52) U.S. Cl. 600/509

(57) ABSTRACT

An electrocardiogram signal converter comprises an electrocardiogram signal detector, a first voltage amplifier, at least one low pass filter, at least one high pass filter, a second voltage amplifier, an optical isolator and an RS232 input/output port. The electrocardiogram signal detector captures an electrocardiogram signal, and the signal is amplified and filtered through the first and the second voltage amplifiers and the high and low pass filter/filters before being transmitted to the optical isolator. Afterward, the output signal of the optical isolator is digitized by the analog-to-digital conversion device and is transmitted to a computer through the RS232 interface for heart rate variability analysis.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention is related to an electrocardiogram signal converter and an analog-to-digital conversion device thereof, and more specifically, they are used for analyzing heart rate variability.

### BACKGROUND OF THE INVENTION

Sympathetic nerves and parasympathetic nerves, both belonging to human autonomic nervous system, are closely related to the daily operation of a human body. Autonomic imbalance may induce various acute and chronic diseases, for example, heart disease, hypertension, etc., and may even lead to a sudden death, if serious. Even healthy individuals suffering from autonomic disorders may have palpitation, dyspnea (shortness of breath), gastrointestinal disorders, insomnia, etc. Hence, the protection for autonomic nervous system is not only an important issue in medicine but also a personal concern to an individual everyday. Our living quality depends on how well our autonomic nervous system functions. Signs and symptoms of certain severe diseases at an early stage are attributable to autonomic disorders. Hence, early detection of autonomic disorder in an individual or a patient is likely to reduce the chance of or even prevent tragedies.

In recent years, plenty of new technologies to evaluate the autonomic functions were successfully developed. Given the sophisticated computer hardware and software know-how available, today we are able to detect and perform quantitative analysis of a person's autonomic cardiac activity in light of the minute fluctuations of hear rate, known as heart rate variability (HRV), taken while the person is at rest. In other words, the new technologies allow us to analyze or evaluate a normal person's autonomic functions without interfering with the person's daily life. Heart rate variability analysis stands out above the other methods for evaluation of autonomic functions, because it has the following advantages: (1) being a non-invasive diagnosis technology, it does not cause a subject any pain, (2) the hardware it uses is cheap, thus it has the potential for large-scale promotion, and (3) many animal tests and human tests prove that it evaluates autonomic functions accurately. Therefore, the technology of heart rate variability analysis is in wide use in recent years, and related research is being conducted on it unceasingly.

The advent of the technology about spectrum analyzers in the early 1980s enabled heart rate variability analysis to be brought into full play, when autonomic functions were quantitatively analyzed in light of the beating cycle of heart. Hence, heart rate variability analysis gradually becomes the best non-invasive method for detecting autonomic functions. With spectrum analysis, researchers discovered that the minute fluctuations of heart rate variability could be definitely divided into two groups, that is, high-frequency (HF) component and low frequency (LF) component. The HF component is synchronous to animals' breath signals, so it is also known as breath component, which occurs approximately every three seconds in a human being. The source of the LF component that takes place approximately every ten seconds in a human being remains unidentified, though researchers infer that they are relevant to vascular motion or baroreflex. Some academics went further to divide the LF component into two categories, that is, very low frequency (VHF) component and low frequency component. At present, many physiologists and cardiologists believe that the HF component or total power (TP) reflects parasympathetic functions, whereas the ratio of LF component to HF component (LF/HF) reflects sympathetic activity. In addition to being an index of autonomic functions, heart rate variability reflects various kinds of information about human bodies, as indicated by some researches. For instance, patients diagnosed with intracranial hypertension usually have relatively low heart rate variability, the death rate of an elder whose LF component of heart rate variability decreases by a standard deviation is 1.7 times that of normal persons, and the LF component of heart rate variability vanishes in a brain-dead person. Furthermore, there are changes in heart rate variability in a patient who exhibits rejection reactions after heart transplantation. During an operation, heart rate variability reflects depth of anesthesia.

The analysis of heart rate variability involves measuring heart beat cycles based on electrocardiogram signals. Hence, both the convenience and the precision of capturing electrocardiogram signals are of vital importance to the popularization of heart rate variability analysis. Conventional hardware used for capturing electrocardiogram signals is rather costly and inconvenient, turning away ordinary users and restraining the promotion and development.

As disclosed in Patent No. 363,404 (corresponding to U.S. Pat. No. 6,360,117) published in the Patent Gazette of the Republic of China (Taiwan), the electrocardiogram signal converter previously invented by the applicant for analyzing heart rate variability allows electrocardiogram signals to be transmitted through an interface compatible with a printer input/output port of a personal computer (PC) or notebook, so the cost of the apparatus for analyzing heart rate variability can be tremendously reduced. However, the application of the aforesaid patent is rather limited, as the technology involves treating the printer input/output port as an interface for transmitting electrocardiogram signals.

### BRIEF SUMMARY OF THE INVENTION

The primary objective of the present invention is to provide an electrocardiogram signal converter intended for analyzing heart rate variability to meet the specifications of standard serial (RS232) interface. To analyze heart rate variability, one only needs to connect the signal output end of the electrocardiogram signal converter to a computer having an RS232 interface and to process electrocardiogram signals with an appropriate analytical program. Accordingly, heart rate variability can be analyzed without using any expensive medicine-oriented computers, so as to significantly reduce the cost.

The RS232, a digital signal transmission standard, has been in wide use for a long time. Although in the latest computers put on the market, the Universal Serial Bus (USB) gradually replaces the RS232, the application of the RS232 remains unchecked in the presence of the USB-RS232 conversion interface. Since the RS232 interfaces are still quite useful today, products having an RS232 interface that functions as a transmission interface are still in wide use. The present invention chooses RS232 as a digital signal transmission interface to transmit electrocardiogram signals. Hence, the present invention may be applied to old-model computers, provided that they are equipped with RS232 interfaces.

The electrocardiogram signal converter put forth in the present invention comprises an electrocardiogram signal detector, a first voltage amplifier, at least one low pass filter, at least one high pass filter, a second voltage amplifier, an optical isolator and a standard serial port (RS232 input/output port). In the form of an electrode, the electrocardiogram signal detector is connected to a person to capture an electrocardiogram signal. The purpose of the first and second voltage amplifiers is to amplify the voltage of the electrocardiogram signal. The purpose of the at least one low pass is to filter high-frequency noise out of the electrocardiogram signal, whereas the at least one high pass filter is to filter low-frequency noise out of the electrocardiogram signal as well as to block direct current (DC). The second voltage amplifier amplifies the voltage of the electrocardiogram signal again, and is coupled with a bias voltage to eliminate noise and adjust the baseline of the waveform of the electrocardiogram signal. The optical isolator receives the output signal from the second voltage amplifier, and the output end and the input end of the optical isolator are completely separated from each other. The analog-to-digital conversion device digitizes the electrocardiogram signal. The RS232 input/output port is connected to the analog-to-digital conversion device, so as to send the electrocardiogram signals converted by the analog-to-digital conversion device to a computer for the heart rate variability analysis.

The above mentioned analog-to-digital conversion device comprises a clock generator, a clock synchronization circuit, an analog-to-digital converter, a chip start signal generation circuit and an RS232 voltage converter. The clock synchronization circuit is coupled with the clock generator to synchronize the clocks generated by the clock generator and the clocks of the RS232 input/output port. The analog-to-digital converter outputs data in series, and digitizes the electrocardiogram signal for being processed and analyzed by a computer. The chip start signal generation circuit provides a start signal to the analog-to-digital converter. The voltage converter converts voltages between the analog-to-digital converter and the RS232 input/output port, in order to meet the voltage requirements of the transistor-transistor logic (TTL) components in the analog-to-digital converter.

The chip start signal generation circuit comprises a flip-flop which presets the start bit of the signal of the RS232 input/output port as the start signal of the analog-to-digital converter. The clock generator is reset at the moment the start signal emerges, so as to synchronize the clocks generated by the clock generator and the clocks of the RS232 input/output port. Upon completion of data transmission, the analog-to-digital converter generates an end signal to clear the start signal.

### BRIEF DESCRIPTION OF THE SEVERAL VIEWS OF THE DRAWINGS

FIG. 1 illustrates the function block diagram of the electrocardiogram signal converter in accordance with the present invention;

FIG. 2 illustrates the schematics of the amplifiers and filters of the electrocardiogram signal converter in accordance with the present invention;

FIG. 3 illustrates the function block diagram of the analog-to-digital conversion device of the electrocardiogram signal converter in accordance with the present invention;

FIG. 4 illustrates the schematics of the analog-to-digital conversion device of the electrocardiogram signal converter in accordance with the present invention; and

FIG. 5 exemplifies an electrocardiogram derived from the electrocardiogram signal converter of the present invention and analysis result thereof.

### DETAILED DESCRIPTION OF THE INVENTION

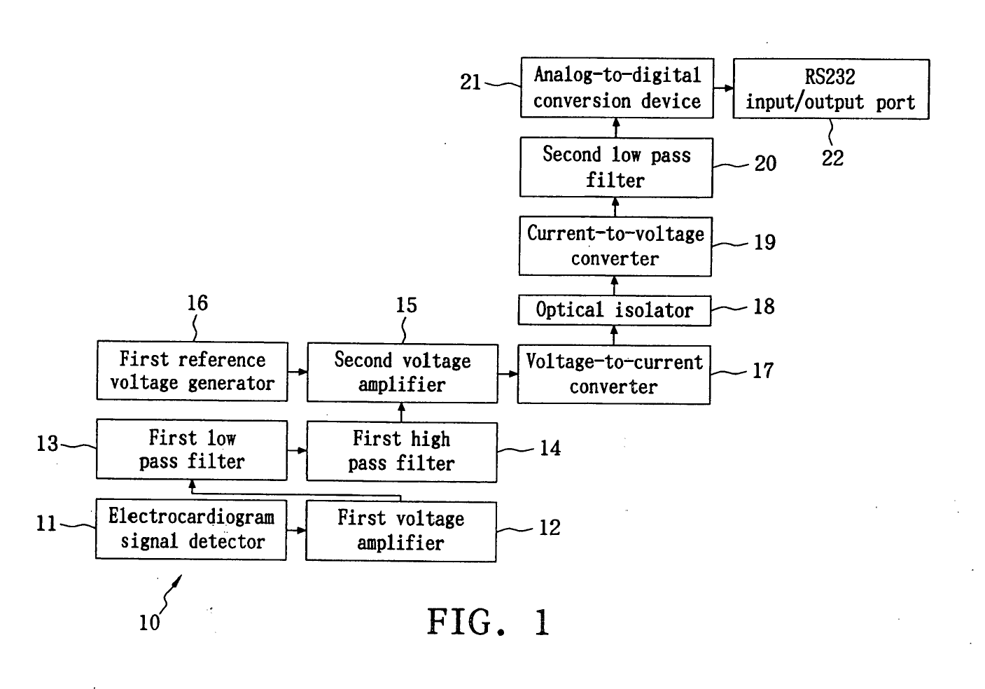

FIG. 1 shows the function block diagram of an electrocardiogram signal converter 10 put forth in the present invention. The electrocardiogram signal converter 10 captures an electrocardiogram signal of a person for heart rate variability analysis. The electrocardiogram signal converter 10 comprises an electrocardiogram signal detector 11, a first voltage amplifier 12, a first low pass filter 13, a first high pass filter 14, a second voltage amplifier 15, a first reference voltage generator 16, a voltage-to-current converter 17, an optical isolator 18, a current-to-voltage converter 19, a second low pass filter 20, an analog-to-digital conversion device 21 and an RS232 input/output port 22. The electrocardiogram signal detector 11 may be constituted of electrodes, and is connected to a person to detect his/her electrocardiogram signals. The electrocardiogram signal detected by the electrocardiogram signal detector 11 undergoes voltage amplification, low-frequency filtration and high-frequency filtration through the first voltage amplifier 12, the first low pass filter 13 and the first high pass filter 14, respectively. Sequentially, the electrocardiogram signal is sent to the second voltage amplifier 15, and subjected to a bias voltage based on the comparison of the voltage of the electrocardiogram signal with a reference voltage from the first reference voltage generator 16, so as to eliminate noise and adjust the baseline of the waveform. Afterward, with the voltage-to-current converter 17, the voltage signal is converted into a current signal for driving the optical isolator 18, and the current signal outputted from the optical isolator 18 is reversely converted by the current-to-voltage converter 19 into a voltage signal. After that, the second low pass filter 20 filters out high-frequency noise from the signal, and the signal is converted into a digital signal by the analog-to-digital conversion device 21. Finally, the digital signal is sent to the RS232 input/output port 22 whereby the electrocardiogram signal can be transmitted for subsequent heart rate variability analysis.

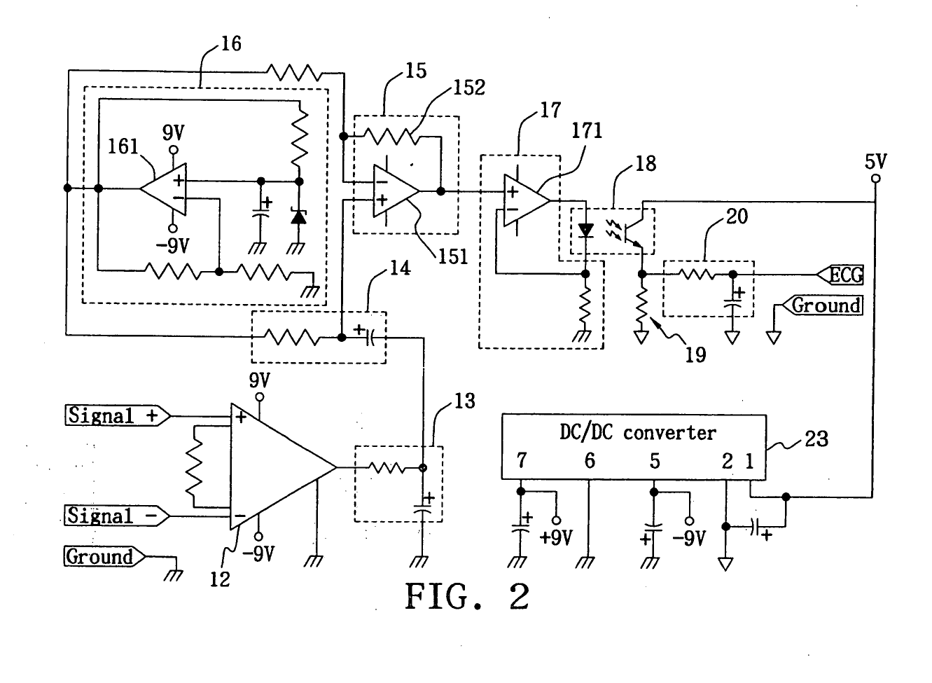

FIG. 2 shows the schematics of individual components except the analog-to-digital conversion device 21 and the RS232 input/output port 22 depicted in FIG. 1. The first amplifier 12 can be an instrumental surveying amplifier, for example the AD620 in the market, which is an equivalent circuit composed of four operational amplifiers. The input end of the first amplifier 12 receives an electrocardiogram signal detected by the electrocardiogram signal detector 11, and the output signal is sent to the second voltage amplifier 15 through the first low pass filter 13 and the first high pass filter 14. The second voltage amplifier 15 is composed of an operational amplifier 151 and a negative feedback resistor 152, in which one input of the second voltage amplifier 15 receives the output signal from the first high pass filter 14, and the other input receives a reference voltage generated by the first reference voltage generator 16 for bias voltage adjustment. The first reference voltage generator 16 and the voltage-to-current converter 17 are essentially constituted of operational amplifiers 161 and 171, respectively. The input of the voltage-to-current converter 17 is connected to the output of the second voltage amplifier 15, whereas the output of the voltage-to-current converter 17 is connected to the optical isolator 18. In practice, the input and the output of the optical isolator 18 are completely separated from each other. In this embodiment, the current-to-voltage converter 19 is a resistor. High-frequency noise is filtered out of the signal by the second low pass filter 20 once again before the signal is sent to the analog-to-digital conversion device 21. The power for the voltage amplifiers 12 and 15 as well as the filters 13, 14 and 20 is supplied by a DC/DC converter 23.

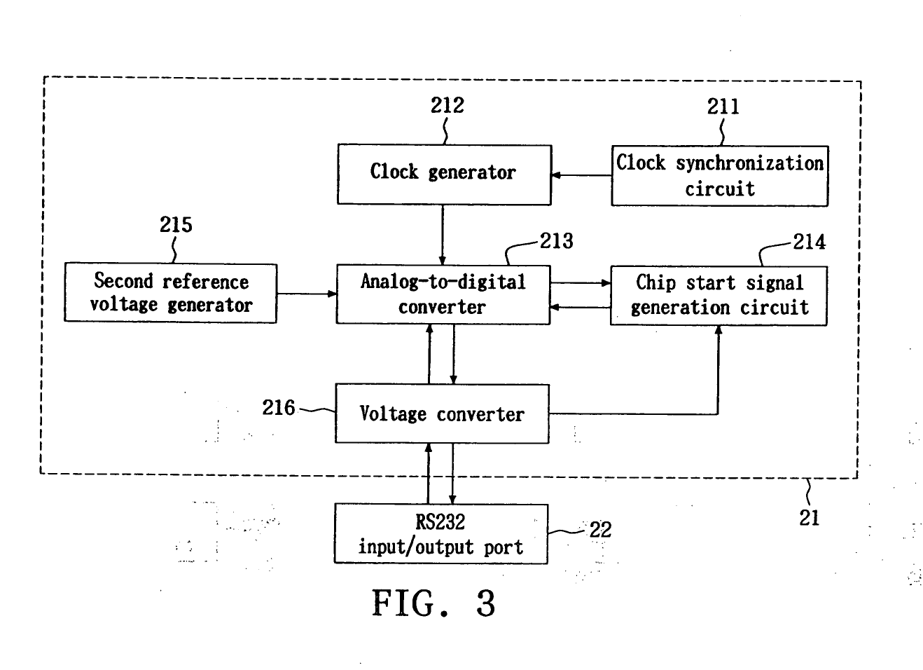

FIG. 3 shows the function block diagram of the analog-to-digital conversion device 21 and the RS232 input/output port 22. The analog-to-digital conversion device 21 comprises a clock synchronization circuit 211, a clock generator 212, an analog-to-digital converter 213, a chip start signal generation circuit 214, a second reference voltage generator 215 and a voltage converter 216. The clock synchronization circuit 211 is coupled with the clock generator 212 to synchronize the clocks generated by the clock generator 212 and the clocks of the RS232 input/output port 22. The analog-to-digital converter 213 digitizes the electrocardiogram signal for being processed and analyzed by a computer. The chip start signal generation circuit 214 provides a start signal for the analog-to-digital converter 213. The voltage converter 216 conducts voltage conversion between the analog-to-digital converter 213 and the RS232 input/output port 22.

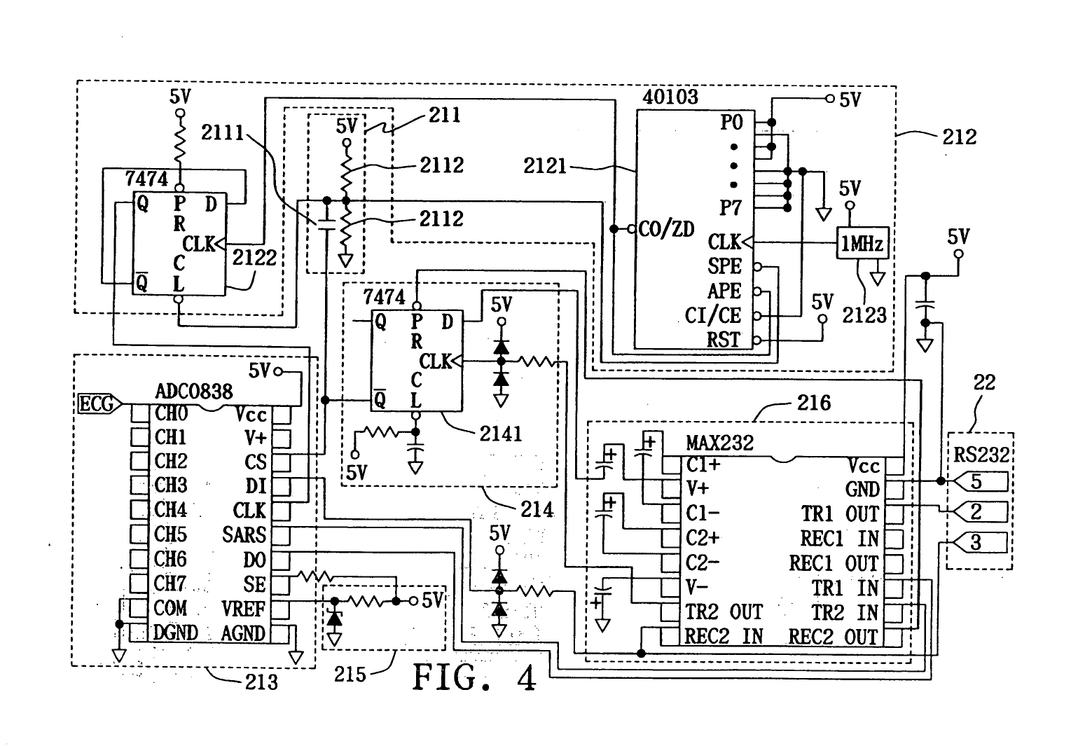

FIG. 4 shows the schematics for the analog-to-digital conversion device 21 and the RS232 input/output port 22. As regards the circuit design of an RS232 interface, synchronization of the clocks that communicate with the hardware on both sides is a challenge indeed. In this embodiment, it is necessary to synchronize the clocks of the hardware, for example a computer, connected to the RS232 input/output port 22 and the clocks of the electrocardiogram signal. In general, the clock frequency of an RS232 interface is derived from the division of 1.8432 MHz. Among the numerous frequency dividers, the CMOS 40103 or 40102 is able to perform 8-bit frequency division, and is powerful and cheap. However, CMOS does have a limitation, that is, it cannot increase the standard operation frequency to 1.8432 MHz. To cope with the frequency problem, in the present invention, the fundamental frequency is set at 1 MHz and a clock synchronization circuit 211 is composed of a capacitor 2111 and two resistors 2112, and is coupled with the clock generator 212 to overcome the problem of clock synchronization. The clock generator 212 is composed of an oscillator 2123, a CMOS 40103 integrated circuit 2121 and a 7474 flip-flop 2122. The oscillator 2123 generates clocks at 1 MHz. The CMOS 40103 integrated circuit 2121 and the 7474 flip-flop 2122 jointly function as a frequency divider to generate symmetric clock square waves as the clocks for the analog-to-digital converter 213. The 40103 integrated circuit 2121 and the 7474 flip-flop 2122 of the clock generator 212 are reset at the moment the rising edge of the serial bit signal is transmitted from the computer to the analog-to-digital converter 213, i.e., the moment the start signal emerges, so as to synchronize the clocks of the analog-to-digital converter 213 and the clocks of the RS232 input/output port 22. As proved by experiment, the above circuit can correctly transmit data even under a baud rate of 38400 bps.

The chip start signal generation circuit 214 essentially consists of a 7474 flip-flop 2141. Basically comprised of the ADC0838 of the National Semiconductor Corporation, the analog-to-digital converter 213 outputs data in a serial manner. First, the start bit of the signal the RS232 input/output port 22 receives from the computer presets the 7474 flip-flop 2141, and the pin Q of the 7474 flip-flop 2141 is connected to the pin CS of the analog-to-digital converter 213. The analog-to-digital conversion of the electrocardiogram signal takes place immediately whenever the pin CS of the analog-to-digital converter 213 receives a command from the computer, and the analog-to-digital converter 213 sends data to the computer as soon as the conversion is finished. Upon completion of the data transmission, the pin SARS of the analog-to-digital converter 213 produces an end signal which undoes the setting of the 7474 flip-flop 2141 in order to clear the signal of the pin CS of the analog-to-digital converter 213. The second reference voltage generator 215 is operative for bias voltage adjustment to the analog-to-digital converter 213. In this embodiment, the voltage converter 216 essentially comprises a MAX232 chip which performs voltage conversion between the analog-to-digital converter 213 and the RS232 input/output port 22, so as to supply appropriate voltages to various components.

Signals of an RS232 interface include, basically, a start bit, data bits and a stop bit. However, in general, there is a usage problem, because no stop bit is automatically created whenever an analog-to-digital converter generates an RS232 transmission signal. The analog-to-digital converter 213 of the present invention can automatically generate a start bit whenever data outputs in series, and the end signal of the pin SARS resets the signal of the pin CS so as to create a stop bit.

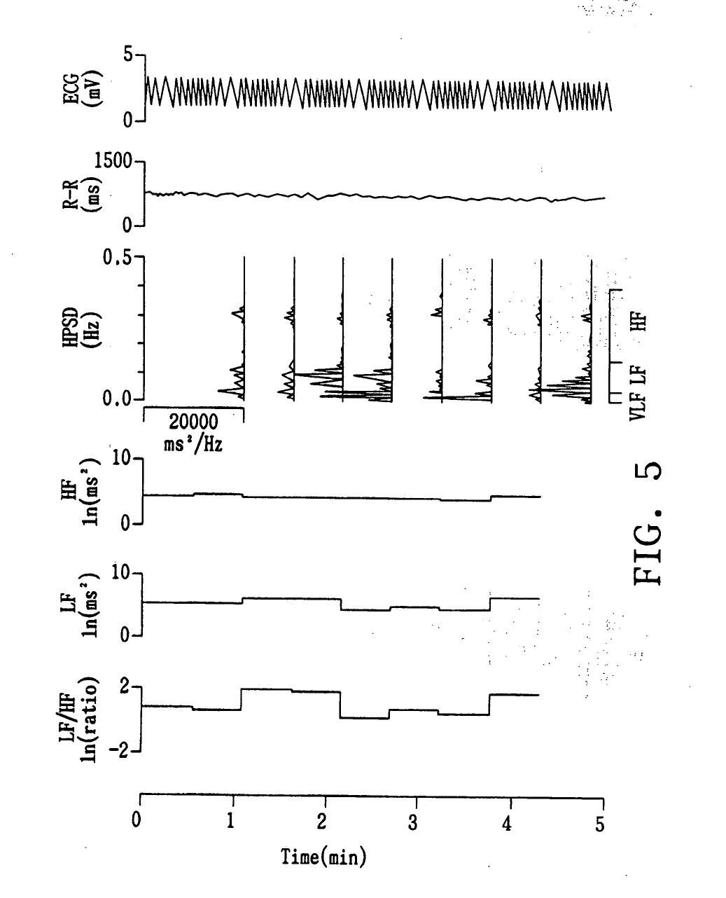

FIG. 5 shows an electrocardiogram that results from a five-minute examination conducted on a person with the electrocardiogram signal converter 10, as well as the result of heart rate variability analysis, in which the R-R represents the interval between heart beats, the ECG represents an electrocardiogram signal, and the HPSD is the heart rate power spectral density of the electrocardiogram.

The above-described embodiments of the present invention are intended to be illustrative only. Numerous alternative embodiments may be devised by those skilled in the art without departing from the scope of the following claims.

1. An electrocardiogram signal converter used for heart rate variability analysis, comprising:
an electrocardiogram signal detector capturing an electrocardiogram signal;
a first voltage amplifier amplifying voltage of the electrocardiogram signal;
at least one low pass filter filtering out high frequency noise of the electrocardiogram signal;
at least one high pass filter filtering out low frequency noise of the electrocardiogram signal and blocking direct current;
a second voltage amplifier amplifying voltage of the electrocardiogram signal again and inputting a bias voltage;
an optical isolator which receives an output signal of the second voltage amplifier;
an analog-to-digital conversion device digitizing an output signal of the optical isolator; and
a standard serial port, RS232 input/output port, connected to the analog-to-digital conversion device and being an interface transmitting the electrocardiogram signal.

2. The electrocardiogram signal converter of claim 1, further comprising a voltage-to-current converter connected to an input end of the optical isolator.

3. The electrocardiogram signal converter of claim 2, wherein the voltage-to-current converter comprises an operational amplifier.

4. The electrocardiogram signal converter of claim 1, further comprising a current-to-voltage converter connected to an output end of the optical isolator.

5. The electrocardiogram signal converter of claim 1, wherein the at least one low pass filter comprises a first low pass filter and a second low pass filter which are ahead and behind electrically connected to the second voltage amplifier, respectively.

6. The electrocardiogram signal converter of claim 1, wherein the at least one high pass filter is electrically disposed between the first and second voltage amplifiers.

7. The electrocardiogram signal converter of claim 1, further comprising a first reference voltage generator for providing a reference voltage to the second voltage amplifier, so as to perform the bias voltage adjustment to the electrocardiogram signal.

8. The electrocardiogram signal converter of claim 1, further comprising a DC/DC converter for providing power to at least one of the first voltage amplifier, the second voltage amplifier, the at least one high pass filter and the at least one low pass filter.

9. The electrocardiogram signal converter of claim 1, wherein the output end and input end of the optical isolator are completely separated.

10. An analog-to-digital conversion device used for performing analog-to-digital conversion to an electrocardiogram signal and transmitting the electrocardiogram signal through a standard serial port, RS232 input/output port, so as to perform a heart rate variability analysis, the analog-to-digital conversion device comprising:
a clock generator generating clocks;
a clock synchronization circuit coupled with the clock generator synchronizing the clocks of the generator and clocks of the RS232 input/output port;
an analog-to-digital converter digitizing the electrocardiogram signal;
a chip start signal generation circuit for providing a start signal of the analog-to-digital converter; and
a voltage converter for converting voltages between the analog-to-digital converter and the RS232 input/output port.

11. The analog-to-digital conversion device of claim 10, further comprising a second reference voltage generator for performing bias voltage adjustment to the analog-to-digital converter.

12. The analog-to-digital conversion device of claim 10, wherein the clock generator comprises at least one oscillator and at least one frequency divider.

13. The analog-to-digital conversion device of claim 10, wherein output data from the analog-to-digital converter is in series.

14. The analog-to-digital conversion device of claim 10, wherein the chip start signal generation circuit comprises a flip-flop which presets a start bit of the signals of the RS232 input/output port as a start signal of the analog-to-digital converter.

15. The analog-to-digital conversion device of claim 14, wherein the analog-to-digital converter generates an end signal for clearing the start signal whenever data is transmitted completely.

16. The analog-to-digital conversion device of claim 14, wherein the clock generator is being reset at the moment when the start signal emerges to synchronize the clocks of the clock generator and the clocks of the RS232 input/output port.

17. The analog-to-digital conversion device of claim 10, wherein the clock synchronization circuit comprises a capacitor and two resistors.
